(19) United States
(12) Patent Application Publication
(10) Pub. No.: US 2003/0220683 A1
(43) Pub. Date: Nov. 27, 2003

(54) Endoluminal device having barb assembly and method of using same

(76) Inventors: Zarouhi Minasian, Bedford, MA (US); James Weldon, Roslindale, MA (US)

Correspondence Address: Christopher R. Lewis, RATNER & PRESTIA, One Westlakes, Berwyn, Suite 301, P.O. Box 980, Valley Forge, PA, 19482-0980, US

(21) Appl. No.: 10/153,351

(22) Filed: May 22, 2002

(51) Int. Cl. A61F 2/06

(52) U.S. Cl. 623/1.15

(57) ABSTRACT

An endoluminal device for implantation in a body lumen reduces movement or migration of the device after implantation by the use of barbs or barb assemblies. A first embodiment uses at least one barb assembly having first and second portions attached to an implant, such as a stent, and at either sides of a bend. The second portion is adapted to protrude radially inward when the implant is in the radially compressed configuration and radially outward when the implant is in its radially expanded configuration. A second embodiment uses a barb having a curved segment which is curved proximally and radially inwardly. A third embodiment utilizes at least one barb assembly having a wire with a length greater than the cell height of the implant across which it extends and a substantially uniform cross-sectional area. Methods for implanting such devices are also contemplated.

### FIELD OF THE INVENTION

This invention relates generally to endoluminal devices, and more particularly concerns implants such as stents and grafts for placement in an area of a body lumen that has been weakened by damage or disease, such as by aneurysms of the abdominal aorta. In particular, the present invention relates to such devices having barbs that engage the body lumen upon or after deployment of the device. The invention also relates to methods for using such barbed endoluminal devices.

### BACKGROUND OF THE INVENTION

A stent is an elongated device used to support an intraluminal wall. In the case of a stenosis, a stent provides an unobstructed conduit through a body lumen in the area of the stenosis. Such a stent may also have a prosthetic graft layer of fabric or covering lining the inside and/or outside thereof. A covered stent is commonly referred to in the art as an intraluminal prosthesis, an endoluminal or endovascular graft (EVG), an endoluminal device, or a stent-graft. As used herein, the term “implant” shall mean any covered stent or uncovered stent or other medical device suitable for implantation in a body and for use in connection with the present invention.

A stent-graft may be used, for example, to treat a vascular aneurysm by removing the pressure on a weakened part of an artery so as to reduce the risk of rupture. Typically, a stent is implanted in a blood vessel at the site of a stenosis or aneurysm endoluminally, i.e. by so-called “minimally invasive techniques” in which the stent, restrained in a radially compressed configuration by a sheath or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means. The term “proximal” as used herein refers to portions of the stent or delivery system relatively closer to the end outside of the body, whereas the term “distal” is used to refer to portions relatively closer to the end inside the body.

When the introducer has been threaded into the body lumen to the stent deployment location, the introducer is manipulated to cause the stent to be ejected from the surrounding sheath or catheter in which it is restrained (or alternatively the surrounding sheath or catheter is retracted from the stent), whereupon the stent expands to a predetermined diameter at the deployment location, and the introducer is withdrawn. Stent expansion may be effected by spring elasticity, balloon expansion, or by the self-expansion of a thermally or stress-induced return of a memory material to a pre-conditioned expanded configuration.

Among the many applications for stent-grafts is that of deployment in lumen for repair of aneurysms, such as abdominal aortic aneurysms (AAA). An AAA is an area of increased aortic diameter that generally extends from just below the renal arteries to the aortic bifurcation. AAA generally results from deterioration of the arterial wall, causing a decrease in the structural and elastic properties of the artery. In addition to a loss of elasticity, this deterioration also causes a slow and continuous dilation of the lumen.

The standard surgical repair of AAA is an extensive and invasive procedure typically requiring a weeklong hospital stay and an extended recovery period. To avoid the complications of the surgical procedure, practitioners commonly resort to a minimally invasive procedure using endoluminal stent-grafts to reinforce the weakened vessel wall, as mentioned above. At the site of the aneurysm, the practitioner deploys the stent-graft, anchoring it above and below the aneurysm to relatively healthy tissue. The anchored stent-graft diverts blood flow away from the weakened arterial wall, minimizing the exposure of the aneurysm to high pressure.

Intraluminal stents for repairing a damaged or diseased artery or to be used in conjunction with a graft for delivery to an area of a body lumen that has been weakened by disease or damaged, such as an aneurysm of the abdominal aorta, are well established in the art of medical science. The use and description of such intraluminal stents are set forth in U.S. Pat. Nos. 5,681,346; 5,800,526; and 5,843,164. These references are each incorporated in their entirety as part of this specification. One aspect of the use of such intraluminal stents are the means by which such devices are secured within the intraluminal body in which they are to be deployed. This is important because subsequent movement of the stent (or “migration”) could cause the aneurysm to become exposed to blood pressure. In particular, if the device migrates proximally over time, a leak at the distal end of the device (i.e., a “type I endoleak”) could cause blood to undesirably flow to the aneurysm.

Stents with fixed barbs have been used to engage the vessel wall as the deployment sheath is pulled back from the stent. However, such stents with fixed integrated barbs are difficult to load into the catheter deployment system. Fixed barbs are not flush to the perimeter of the stent and therefore have a tendency to prevent the stent from being loaded or to cause the stent to become lodged inside the catheter during loading. Moreover, catheter deployment systems used to deploy stents with barbs are commonly scratched during the deployment of the stent. Scratching of the catheter deployment system can cause plastic particulate from the catheter deployment system to enter the bloodstream, potentially forming an embolus.

Accordingly, it can be seen that while the art has advanced the use of barbs to minimize migration of a deployed stent-graft, such barbs bring with them additional or new problems such as damaging the wall of the vessel or hindering the placement of the stent and body graft. While the art has attempted to address such problems, there still remains a need for improvement in the art. Such improvement is critical inasmuch as scratching of the deployment system can cause plastic or other particulate from the deployment system to enter the blood stream, potentially forming an embolus.

### SUMMARY OF THE INVENTION

In view of its purposes and the needs of the prior art, the present invention provides an endoluminal device comprising an implant and a barb or barb assembly. According to a first embodiment, a device for implantation in a body lumen comprises an implant and at least one barb assembly. The implant may be a stent having a radially compressed configuration and a radially expanded configuration and comprising at least one filament which pivots as the stent moves between the radially compressed configuration and the radially expanded configuration. The barb assembly comprises: (i) a first portion attached to the stent, (ii) a bend, and (iii) a second portion, disposed opposite the first portion relative to the bend and having a bearing surface. The second portion is adapted to protrude radially inward when the stent is in the radially compressed configuration. The filament radially contacts and imparts a radially outward force against the bearing surface as the stent moves from the radially compressed configuration to the radially expanded configuration to cause the second portion to protrude radially outward (or “flip” outwardly) when the stent is in its radially expanded configuration. A method for implanting an endoluminal device according to this first embodiment in a body lumen comprises the steps of compressing the endoluminal device into a radially compressed configuration and retaining the device in an introducer; introducing the introducer into the body lumen to a deployment location; and deploying the endoluminal device from the introducer and into the body lumen.

According to a second embodiment of the present invention, a device for implantation in a body lumen from a proximal access location comprises an implant and at least one barb. The implant may be a stent having a radially compressed configuration for insertion into a sheath and comprising at least one filament. The barb comprises (i) a base segment attached to the filament and (b) a curved segment extending from the base segment and terminating in a point. The curved segment is curved proximally and radially inwardly but not to such an extent so as to extend radially within the periphery defined by the stent. A method for implanting an endoluminal device according to this embodiment in a body lumen comprises the steps of compressing the endoluminal device into a radially compressed configuration and retaining the device in an introducer; introducing the introducer into the body lumen to a deployment location; deploying the endoluminal device from the introducer and into the body lumen; and twisting the implant between 1 and 15 degrees to cause the curved segment to engage the body lumen.

According to a third embodiment of the present invention, a device for implantation in a body lumen from a proximal access location comprises an implant and at least one barb assembly. The implant may be a stent having a radially compressed configuration and a radially expanded configuration and defining a plurality of cells each having a cell height. The barb assembly comprises: (i) a wire extending from the top of a cell to the bottom of a cell and having a length greater than the cell height and a substantially uniform cross-sectional area; and (ii) a hook attached to the wire and extending radially outward. The wire is formed to arc radially inwardly when the stent is in its radially compressed configuration and is capable of being arced radially outward when the stent is in its radially expanded configuration. A method for implanting an endoluminal device according to this embodiment in a body lumen comprises the steps of compressing the endoluminal device into a radially compressed configuration and retaining the device in an introducer; introducing the introducer into the body lumen to a deployment location; deploying the endoluminal device from the introducer and into the body lumen; and imparting a radially outward force against the barb assembly to cause the barb assembly to arc radially outwardly and cause the hook to engage the body lumen.

The foregoing general description and subsequent detailed description are representative, not restrictive, of the invention.

### BRIEF DESCRIPTION OF THE DRAWING

The invention is best understood when the following detailed description is read with reference to the attached drawing, in which:

FIG. 1 depicts a view of a portion of an endoluminal device according to a first embodiment of the present invention;

FIG. 2 depicts an enlarged portion of the device shown in FIG. 1 and shows a barb assembly according to the present invention;

FIG. 3a depicts a perspective view of a portion of the device shown in FIG. 1 in its radially expanded configuration and shows a barb assembly according to the first embodiment of the present invention;

FIG. 3b depicts a perspective view of a portion of the device shown in FIG. 1 in its radially compressed configuration and shows a barb assembly according to the first embodiment of the present invention;

FIG. 4a depicts view of a portion of an endoluminal device according to a second embodiment of the present invention;

FIG. 4b depicts a top view of the device shown in FIG. 4a in its radially expanded and engaged configuration;

FIG. 4c depicts a top view of the device shown in FIG. 4a in its radially compressed configuration;

FIG. 5a depicts a view of a portion of an endoluminal device according to a third embodiment of the present invention;

FIG. 5b depicts a side view along the lines A-A of a portion of the device shown in FIG. 5a in its radially expanded configuration and shows a barb assembly according to the third embodiment of the present invention; and

FIG. 5c depicts a side view along the lines A-A of a portion of the device shown in FIG. 5a in its radially compressed configuration and shows a barb assembly according to the third embodiment of the present invention.

### DETAILED DESCRIPTION OF THE INVENTION

The invention will next be illustrated with reference to the figures wherein the same numbers indicate similar elements in all figures. Such figures are intended to be illustrative rather than limiting and are included herewith to facilitate the explanation of the apparatus of the present invention.

The present invention is directed to devices for implantation in a body lumen. Such devices include an endoluminal device used to treat an Abdominal Aortic Aneurysm (AAA). Such an endoluminal device typically comprises a stent having a graft extending along a portion of the stent. Devices according to the present invention may also include other implants which have a stent-like structure and, after implantation of which, migration is sought to be minimized. The body lumen in which a device of the present invention may by implanted include any body lumen in which such devices are typically implanted to perform a wide range of medical functions. In the AAA application, the body lumen is at least one artery, such as the aorta or the aorta and one or both iliac arteries.

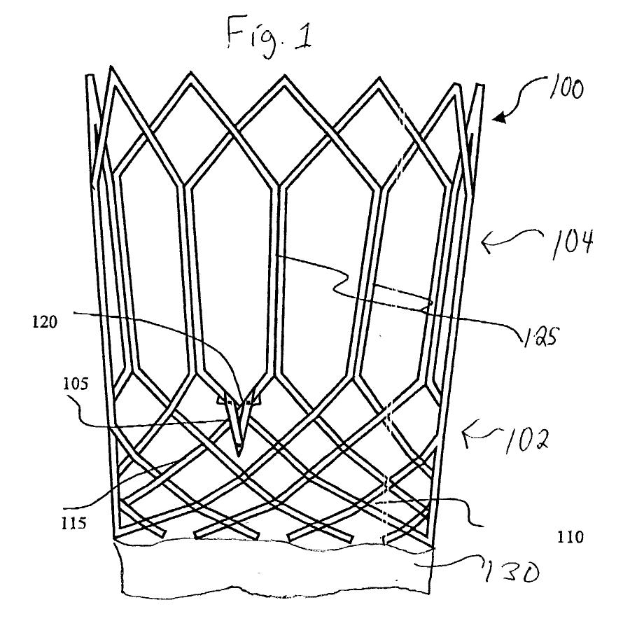

The device of the present invention uses an implant and a barb or barb assembly. The implant used in the present invention can be any number of suitable stents known in the art. A number of suitable stent configurations are described and referenced in co-pending U.S. patent application Ser. No. 09/442,165, entitled MULTI-SECTION FILAMENTARY ENDOLUMINAL STENT, assigned to the assignee of this application and incorporated herein by reference. The stent may be wound, braided, or made from a laser-cut tube. The stent may be self-expanding or may be capable of expansion by an external force, such as a balloon. The material of the stents may also be any suitable material typically used for such applications, such as nitinol. In the embodiments discussed, the stent has a braided section 102 and a wound section 104, as shown for example in FIG. 1. In the embodiments described, each stent has a radially compressed configuration suitable for loading into an introducer and a radially expanded configuration which it assumes or is caused to assume upon deployment in a body lumen. Also, the stents described herein have a filament, which can be a wire, strand, or a remaining portion from a laser-cut tube.

FIG. 1 depicts a device according to a first embodiment of the present invention. FIG. 1 shows an expanded filamentary stent 100 having a braided section 102 and a would section 104, as is described in the '165 application. Stent 100 comprises a first filament 110 and a second filament 115, both of which extend along both braided section 102 and wound section 104. Within the wound section, a plurality of hexagonal cells 125 (also referred to herein as “vertical cells”) are formed by the filaments, with each cell having a base defined by two segments of the hexagonal cell. First filament 110 and second filament 115 also form a plurality of intersections, such as intersection 120, defined by the two filaments crossing one another.

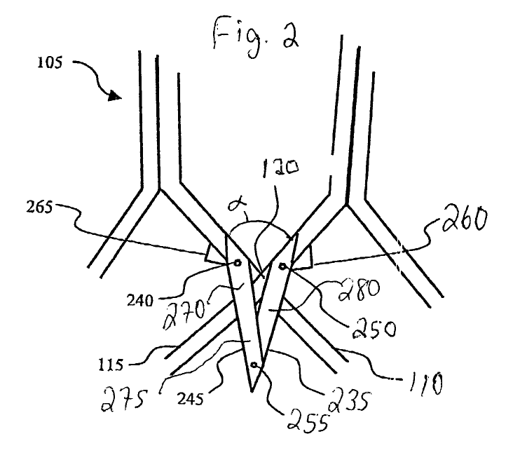

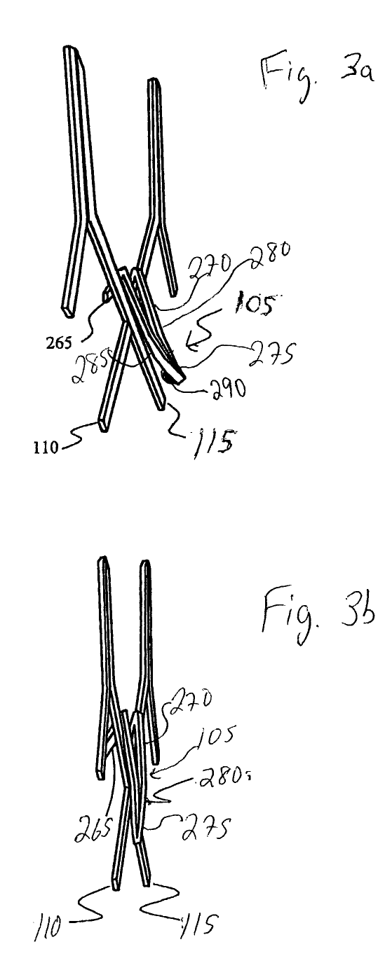

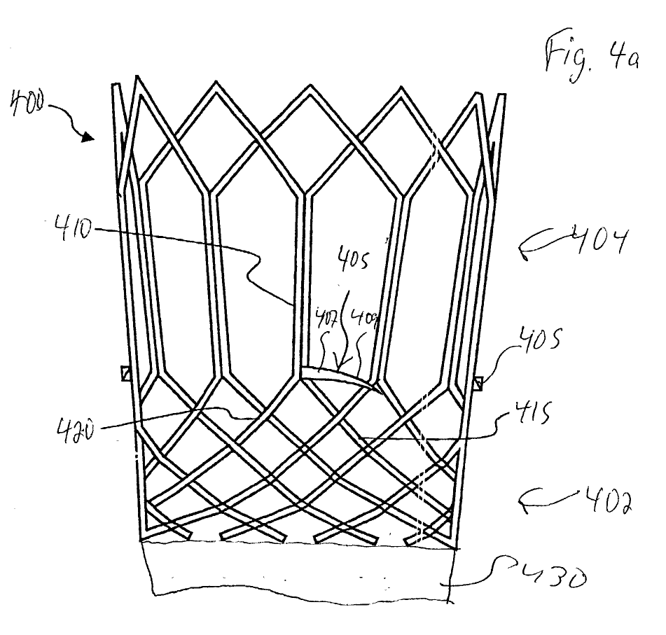

The device shown in FIG. 1 also includes a self-deploying barb assembly 105, which is attached to stent 100 adjacent intersection 120. FIGS. 2, 3a, and 3b show self-deploying barb assembly 105 in more detail. As shown therein, self-deploying barb assembly 105 comprises: (i) a first portion 270 attached to the stent, (ii) a bend 280, and (iii) a second portion 275, disposed opposite the first portion from the bend and having a bearing surface 285. Bearing surface 285 is the underside of second portion 275, as viewed in FIG. 2. Barb assembly includes a first wire 235 and a second wire 245, each of which extending across first portion 270 and second portion 280 and each having a bend 275. As shown in these figures, a first end of first wire 235 and a first end of second wire 245 are disposed within first portion 270 and are attached to stent 100. The other ends of the two wires are attached to one another to form a point. More specifically, second wire 245 is attached to first filament 110 and first wire 235 is attached to second filament 115 in the area of intersection 120. A wide variety of ways to attach the wires to the filaments may be employed, e.g. welding, suturing, gluing, and the like, so long as the means for attachment do not adversely affect the biocompatibility of the stent.

Self-deploying barb assembly 105 is pre-fabricated and made of a biocompatible wire, such as nitinol or a material compatible with the biocompatible material of stent 100. In this particular example, self-deploying barb assembly 105 is in the area of intersection 120, which is in a row of stent 100 between braided section 102 and wound section 104. More specifically, barb assembly 105, including bend 120, is disposed adjacent intersection 120. The present invention is not limited to this configuration. Self-deploying barb assemblies 105 may also be fixed to vertical cell segments 125 or to another row within braided section 102. Stent 100 may include a plurality of self-deploying barb assemblies 105 attached along the perimeter of stent 100 and having variable dimensions and geometry, as long as both stent 100 and self-deploying barb assemblies 105 function within a medically acceptable tolerance.

In some embodiments, the device may also include a graft 130 as shown in FIG. 1. Such grafts may be used in an endoluminal device for treating AAA. Grafts serve to prevent blood from flowing across the device to an aneurysm sac. The material for such grafts may be any suitable material used for such purposes, and the graft may be a braided or non-braided graft, and may comprise any graft material known in the art. Suitable graft materials include, but are not limited to, polyethyleneterepthalate (PET), polyetheretherketone (PEEK), polysulfone, polytetrafluroethylene (PTFE), expanded polytetrafluroethylene (ePTFE), fluorinated ethylene propylene (FEP), polycarbonate urethane, a polyolefin (such as polypropylene, polyethylene, or high density polyethylene (HDPE)), silicone, and polyurethane. Preferably, and as shown in FIG. 1, graft 130 is affixed to stent 100 at an area remote from (i.e., axially distant from) barb assembly 105. Typically, the portion where the barbs are located are intended to be placed in the body lumen at a location where there is healthy tissue; on the other hand, a graft is located at a position along the device corresponding to an unhealthy portion of the body lumen, such as an aneurysm sac.

FIG. 2 shows self-deploying barb assembly 105 in more detail including first flat wire 235, a first wire hinge 240, second flat wire 245, a second wire hinge 250, an apex weld 255, a first posterior tab 260, and a second posterior tab 265. Apex weld 255 joins first flat wire 235 to overlapping second flat wire 245, as mentioned above. To prepare the device, self-deploying barb assembly 105 is typically pre-fabricated from a suitable material, such as spring steel, nitinol, or other suitable metals. The assembly is then affixed to first filament 110 and to second filament 115 using first wire hinge 240 and second wire hinge 250, respectively, in the area where first filament 110 and second filament 115 form intersection 120. According to an embodiment of the invention, a first posterior tab 260 and a second posterior tab 265 limit rotation of the hinge on self-deploying barb assembly 105, causing the barb to engage as the diameter of stent 100 changes upon expansion.

FIG. 3a shows a three-dimensional view of a segment of the device of FIGS. 1 and 2 including stent 100, comprising first filament 110 and second filament 115, with the device in its radially expanded configuration. Also shown is an engaged barb assembly 105. When the diameter of stent 100 is increased, the forces exerted on barb assembly 105 cause it to flip from a sub-surface profile in a generally outward direction relative to an axis of stent 100 to engage the vessel wall, as discussed in more detail below. As used herein, the term “engage” means when a portion of the barb assembly protrudes into and contacts the body lumen in a way which decreases migration of the device relative to the body lumen.

FIG. 3b is a three-dimensional view of a segment of the device of FIGS. 1 and 2 including a stent 100 comprising first filament 110 and second filament 115, and an unengaged self-deploying barb 105. When stent 100 is compressed in the deployment catheter, it is formed to be biased in a radially inward direction relative to an axis of stent 100, and thereby preventing the point of barb assembly 105 from scratching the catheter wall.

As can be seen when comparing FIGS. 3a and 3b, second portion 275 of barb assembly 105 (i.e., that portion below the bend 280) swings radially outward to engage the lumen wall as stent 100 radially expands. Thus, second portion 275 is adapted to protrude radially inward when stent 100 is in its radially compressed configuration. This can be done in any number of ways, such as by using a shape memory alloy, such as nitinol which could be configured to have the desired shape in the radially compressed configuration. Spring steel or other metals could also be used. Barb assembly 105 is caused to take its shape as shown in FIG. 3a due to a filament or intersection radially contacting and imparting a radially outward force against bearing surface 285 of the barb assembly 105. More specifically, the radially outward force from stent 100, as it moves from its radially compressed configuration to its radially expanded configuration, is preferably directed somewhere on the bearing surface 285 of second portion 275. To facilitate this extension of barb assembly, it is desirably to cause the force be directed to the end of the second portion furthest from bend 280.

As is known, the angle of some intersections of certain types of stents changes as the stent moves from a radially compressed configuration to a radially expanded configuration. This is true for braided stents or braided portions of stents, such as braided portion 102, in which angle α is shown in FIG. 2. This means that, as stent 100 expands, first filament 110 and second filament 115 swing relative to one another as angle α increases. Thus, the swinging of second filament 115 against bearing surface 285 of second portion 280 can enhance the radial expansion of barb assembly 105 in concert with the radially outward force caused by the expanding stent generally. Preferably, a protuberance 290 is formed on the radially inner side of second portion 280 for abutting against stent 100 as the stent moves between the radially compressed configuration and the radially expanded configuration. Such a protuberance is located at a position such that a filament crosses and contacts the protuberance during radial expansion of the stent.

A method for implanting an endoluminal device in a body lumen involves first compressing the endoluminal device into a radially compressed configuration and retaining it in an introducer. Such an introducer may be a delivery catheter as are well known in the art, such as those described in U.S. patent application Ser. No. 09/573,273, entitled STENT DELIVERY SYSTEM FOR PREVENTION OF KINKING, AND METHOD OF LOADING AND USING SAME, assigned to the assignee of this application and incorporated herein by reference. Next, the introducer is introduced or threaded into the body lumen via a vascular access site to a deployment location, such as by using a well-known percutaneous cut-down technique referred to above. Examples of the vascular access site include the femoral artery. The access site may be surgically exposed and punctured with, for example, an 18-gauge needle. Then, the device is deployed from the introducer and into the body lumen. This is typically done by first aligning the distal end of the device, then retracting an outer sheath of the introducer. After or upon deployment, the endoluminal device expands to form a radial expanded portion and the at least one filament radially contacts the second portion and imparts a radially outward force against the bearing surface as the implant (e.g., stent) moves from its radially compressed configuration to its radially expanded configuration to cause the second portion to protrude radially outward and engage the body lumen when the stent is in its radially expanded configuration. In the event that the stent is self-expanding, the radial expansion of the stent is caused by the removal of the stent from the introducer. On the other hand, if the stent is not self-expanding, the radial expansion of the stent is caused by expanding a balloon (or some other external source of radially outward force) from within the stent.

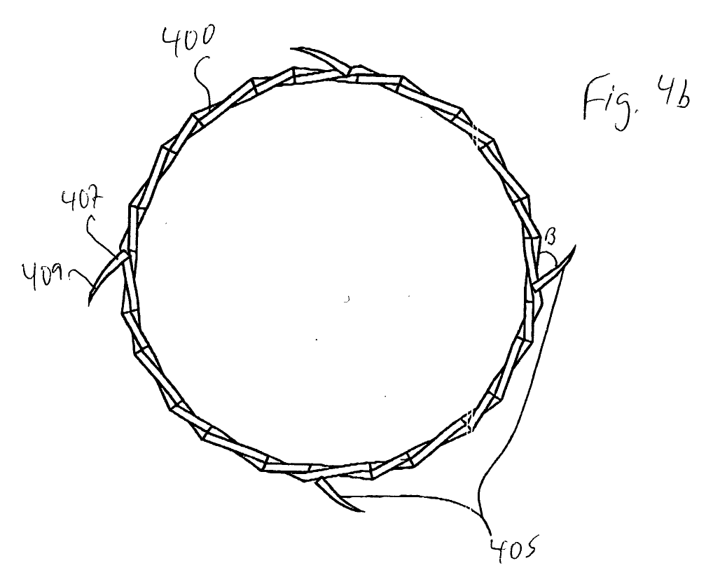

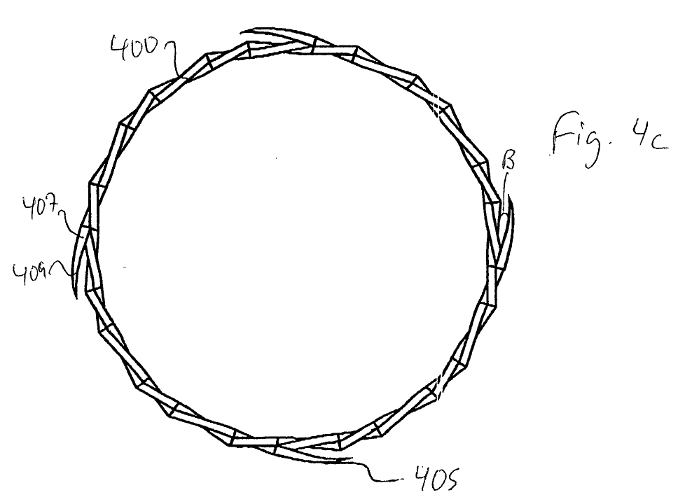

According to another embodiment of the present invention, FIG. 4a shows a device comprising a filamentary stent 400 and a corkscrew barb 405. The stent is similar to stent 105 shown in FIG. 1 in that it has a braided section 402 and a wound section 404. As discussed in connection with the first embodiment, a vertical segment 410, a first filament 415, and a second filament 420 are shown. The barb 405 comprises (i) a base segment 407 attached to one or more filaments (including an intersection)t and (b) a curved segment 409 extending from the base segment and terminating in a point. The curved segment is curved proximally and radially inwardly but not extending radially within the periphery defined by said stent. The downward curvature of barb 405 is shown in FIG. 4a while the radially inward curvature is shown in FIGS. 4b and 4c.

Barb 405 is a biocompatible material, such as nitinol or a material compatible with the biocompatible material of stent 400. Barb 405 is preferably welded at the base of vertical segment 410 where first filament 415 and second filament 420 intersect. Barb 405 is corkscrewed to the longitudinal axis of stent 400. The degree of skewness can range from a small degree to a large degree. The degree of skewness, of course, should be sufficient to allow the barb to hold the stent in place, without causing any damage to the introducer. Preferably, the longitudinal axis of base segment 407 is at least somewhat parallel, more preferably about parallel, to a line intersecting the longitudinal axis at a right angle (90 degrees). When the proximal end of stent 400 is deployed, stent 400 may be rotated to implant barbs 405 into the vessel wall, thereby securing the vessel wall to the stent graft. Barbs 405 are preferably configured such that only a slight rotation of the catheter (e.g., about 15° or less) is required to twist the barbs into the vessel wall. As in the first embodiment, the device may further comprise a graft 430 which is affixed to stent 400 remote from barb 405.

FIG. 4b shows filamentary stent 400 with a plurality of corkscrewed barbs 405. Barbs 405 are pointing in an outward direction, i.e., as they would point in a deployed configuration. This is after the device has been deployed and twisted in the body lumen to cause an increase in angle β.

FIG. 4c shows the compressed filamentary stent 400 with a plurality of corkscrewed barbs 405. When stent 400 is compressed for loading into the stent deployment catheter, barbs 405 are aligned so that the points of barbs 405 do not scrape the inner surface of the outer sheath. Barbs 405 are preferably just slightly curved, as shown in FIG. 4c, as further precaution that the points do not scratch the sheath.

A method to deploy a stent according to this embodiment of the invention again involves compressing the endoluminal device into a radially compressed configuration and retaining the device in an introducer; introducing the introducer into the body lumen to a deployment location; and deploying the endoluminal device from the introducer and into the body lumen. This method also involves twisting the stent between 1 and 15 degrees to cause the curved segment to engage the body lumen. This twisting or rotation involves rotation in an engaging direction. Similarly, if it is desired to disengage the implant, then rotation in the opposite direction would disengage the engagement means.

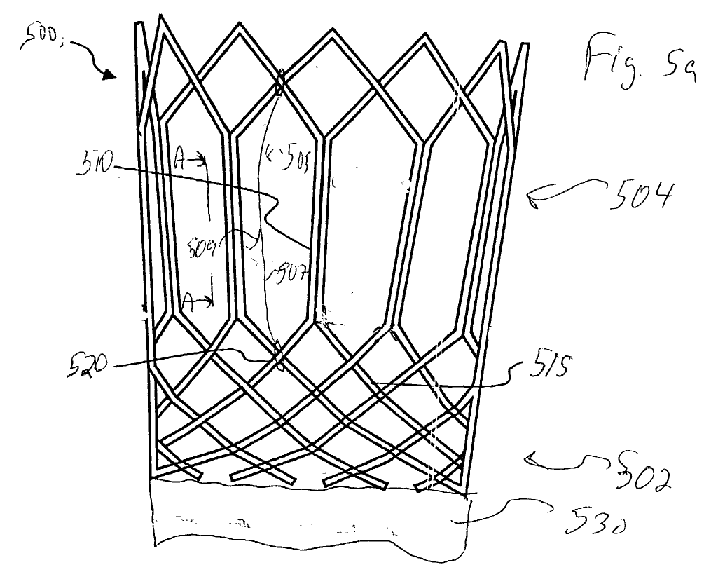

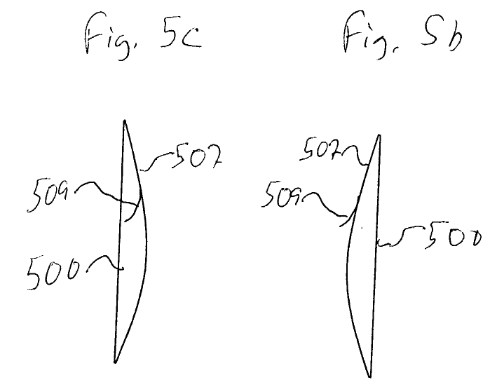

According to another embodiment of the present invention, FIG. 5a shows a device comprising a filamentary stent 500 and a barb assembly 505. The stent is similar to stent 105 shown in FIG. 1 in that it has a braided section 502 and a wound section 504. As discussed in connection with the first embodiment, a vertical segment 510, a first filament 515, and a second filament 520 are shown. The barb assembly comprises: (i) a wire 507 extending from the top of a cell to the bottom of a cell and having a length greater than the cell height and a substantially uniform cross-sectional area and (ii) a hook 509 affixed to the wire and extending radially outward. The term substantially uniform is intended to mean that there is not a change in cross sectional area of greater than 10% and there are no step changes in cross sectional area. The wire is formed to arc radially inwardly, as shown in FIG. 5c, when the stent is in its radially compressed configuration and is capable of being arced radially outwardly, as shown in FIG. 5b, when the stent is in its radially expanded configuration.

The mechanism can involve using stent wires (or ribbon) such that there are two support wires of the same length, on either side of a third wire of a longer length than the supports. As a result the longer wire is bowed and can be placed on the inner or outer side of the stent by pushing on the bowed wire. An illustrative example of such apparatus is depicted in the FIGS. 5a-5c, but the embodiment is not limited thereby. Preferably in this embodiment, the barb assembly is attached at a point where the cell height remains fairly constant as the device is radially expanded. This is generally true for the vertical segments 510 of the wound section 504 of stent 500. In addition, a graft 530 may be included in the device but is preferably remote from barb assembly 505.

The hook(s)/barb(s) can be cut, etched, or attached to the longer wire in any way (facing up, down or both). The barbs can be set on the inner side of the stent for loading and deployment. Then, to deploy the barbs to the outer side post implantation of the device a balloon can be inflated or an inner member dilator/sheath on the delivery system can be advanced in the barb area to push or set the barbs to the outer side of the stent.

A method to deploy a device according to this embodiment of the invention again involves compressing the endoluminal device into a radially compressed configuration and retaining the device in an introducer; introducing the introducer into the body lumen to a deployment location; and deploying the endoluminal device from the introducer and into the body lumen. This method also involves imparting a radially outward force against the barb assembly to cause the barb assembly to arc radially outwardly and cause the hook to engage the body lumen.

In connection with any of the embodiments discussed herein, radiopaque markers may be used in the construction of the attachment means. Such markers assist in deploying, moving or removing the stent since the status of the barb can be determined. Preferably, radiopaque material can be used in the construction of the engagement means, thereby permitting the artisan to further reduce the risk of damage.

In another embodiment of the present invention, the barbs are supported such that during loading into the catheter, in the fully loaded state and during deployment there is no contact between the barbs and the catheter wall. Then, either once the barbed area is exposed or the entire stent-graft system is deployed, the barbs are deployed into place by means such as inflating a balloon or advancing a dilator to push the barbs out into place.

Although illustrated and described herein with reference to certain specific embodiments, the present invention is nevertheless not intended to be limited to the details shown. Rather, various modifications may be made in the details within the scope and range of equivalents of the claims and without departing from the spirit of the invention.

What is claimed is:

1. A device for implantation in a body lumen comprising:
an implant having a radially compressed configuration and a radially expanded configuration and comprising at least one filament which pivots as said implant moves between said radially compressed configuration and said radially expanded configuration; and
at least one barb assembly comprising: (i) a first portion attached to said implant, (ii) a bend, and (iii) a second portion, disposed opposite said first portion from said bend and having a bearing surface, wherein said second portion is adapted to protrude radially inward when said implant is in said radially compressed configuration and said at least one filament radially contacts and imparts a radially outward force against said bearing surface as said implant moves from said radially compressed configuration to said radially expanded configuration to cause said second portion to protrude radially outward when said stent is in said radially expanded configuration.

2. The device of claim 1, wherein:
said barb assembly comprises a first wire and a second wire;
a first end of said first wire and a first end of said second wire are disposed within said first portion and are attached to said stent; and
the other end of said first wire is attached to the other end of said second wire to form a point in said second portion which translates radially outward away from said device as said stent radially expands.

3. The device of claim 2, wherein:
said implant comprises a stent;
said stent comprises a first filament and a second filament;
said first wire is attached to said first filament; and
said second wire is attached to said second filament.

4. The device of claim 3, wherein:
said stent comprises at least one braided section and at least one wound section, which is connected to said braided section; and
said barb assembly is attached at a row in said stent between said braided section and said wound section.

5. The device of claim 4, wherein:
said wound section defines a plurality of hexagonal cells; and
said barb assembly is attached to the base of one of said hexagonal cells.

6. The device of claim 1, wherein said barb assembly comprises a protuberance on the radially inner side of said second portion for abutting against said implant as said implant moves between said radially compressed configuration and said radially expanded configuration.

7. The device of claim 1, wherein said implant comprises a stent, and said stent comprises a plurality of intersections defined by said first filament crossing said second filament and said barb assembly is disposed adjacent a first of said intersections and said bend is located adjacent said first of said intersections.

8. The device of claim 1 wherein said implant comprises a stent and, and further comprises a graft affixed to said stent remote from said barb assembly.

9. A method for implanting an endoluminal device in a body lumen comprising the steps of:
compressing the endoluminal device into a radially compressed configuration and retaining said device in an introducer, wherein said device comprises: (a) an implant comprising at least one filament which pivots as said implant moves between said radially compressed configuration and a radially expanded configuration and (b) at least one barb assembly comprising: (i) a first portion attached to said implant, (ii) a bend, and (iii) a second portion, disposed opposite said first portion from said bend and having a bearing surface and adapted to protrude radially inward when said implant is in said radially compressed configuration;
introducing said introducer into the body lumen to a deployment location; and
deploying said endoluminal device from said introducer and into the body lumen, wherein said endoluminal device expands to form a radial expanded portion and said at least one filament radially contacts said second portion and imparts a radially outward force against said bearing surface as said implant moves from said radially compressed configuration to said radially expanded configuration to cause said second portion to protrude radially outward and engage said body lumen when said stent is in said radially expanded configuration.

10. The method of claim 9, wherein said implant comprises a stent, said stent is self-expanding, and the radial expansion of said stent is caused by the removal of said stent from said introducer.

11. The method of claim 9, wherein said implant comprises a stent and the radial expansion of said stent is caused by expanding a balloon from within said stent.

12. A device for implantation in a body lumen from a proximal access location comprising:
an implant having a radially compressed configuration for insertion into a sheath and comprising at least one filament;
at least one barb comprising (i) a base segment attached to said at least one filament and (b) a curved segment extending from said base segment and terminating in a point, wherein said curved segment is curved proximally and radially inwardly but not extending radially within the periphery defined by said implant.

13. The device of claim 12, wherein said barb is effectively configured such that a rotation of about 15 degrees or less of said device causes the barbs to become affixed into a body lumen.

14. The device of claim 12, wherein:
said implant comprises a stent;
said stent comprises at least one braided section and at least one wound section, which is connected to said braided section; and
said barb is attached to said stent in said wound section.

15. The device of claim 14, wherein:
said at least one filament comprises a first filament and a second filament;
said first filament abuts against said second filament in said wound section to form an intersection adjacent said braided section; and
said barb is attached to said stent at said intersection.

16. The device of claim 12, wherein said implant comprises a stent and the device further comprises a graft affixed to said stent remote from said barb.

17. A method for implanting an endoluminal device in a body lumen comprising the steps of:
compressing the endoluminal device into a radially compressed configuration and retaining said device in an introducer, wherein said device comprises: (a) an implant comprising at least one filament and (b) at least one barb comprising: (i) a base segment attached to said at least one filament and (b) a curved segment extending from said base segment and terminating in a point, wherein said curved segment is curved proximally and radially inwardly but not extending radially within the periphery defined by said implant;
introducing said introducer into the body lumen to a deployment location;
deploying said endoluminal device from said introducer and into the body lumen, wherein said endoluminal device expands to form a radial expanded portion and said at least one curved portion translates away from said implant; and
twisting said stent between 1 and 15 degrees to cause said curved segment to engage said body lumen.

18. A device for implantation in a body lumen comprising:
an implant having a radially compressed configuration and a radially expanded configuration and defining a plurality of cells each having a cell height; and
at least one barb assembly comprising: (i) a wire extending from the top of a cell to the bottom of a cell and having a length greater than the cell height and a substantially uniform cross-sectional area and (ii) a hook attached to said wire and extending radially outward, wherein said wire is formed to arc radially inwardly when said implant is in said radially compressed configuration and is capable of being arced radially outward when said implant is in said radially expanded configuration.

19. The device of claim 18, wherein:
said implant comprises a stent;
said stent comprises at least one braided section and at least one wound section, which is connected to said braided section; and
said barb assembly is attached at a row in said wound section.

20. The device of claim 19, wherein:
said wound section defines a plurality of hexagonal cells; and
said barb assembly is attached to the base and top of one of said hexagonal cells.

21. The device of claim 18, wherein said implant comprises a first filament and a second filament and a plurality of intersections defined by said first filament crossing said second filament, and said wire of said barb assembly is attached to adjacent intersections.

22. The device of claim 18, wherein said implant comprises a stent and the device further comprises a graft affixed to said stent remote from said barb assembly.

23. A method for implanting an endoluminal device in a body lumen comprising the steps of:
compressing the endoluminal device into a radially compressed configuration and retaining said device in an introducer, wherein said device comprises: (a) an implant having a radially expanded configuration and defining a plurality of cells each having a cell height and (b) at least one barb assembly comprising: (i) a wire extending from the top of a cell to the bottom of a cell and having a length greater than the cell height and a substantially uniform cross-sectional area and (ii) a hook attached to said wire and extending radially outward, wherein said barb assembly is formed to arc radially inwardly when said implant is in said radially compressed configuration and is capable of being arced radially outward when said implant is in said radially expanded configuration;
introducing said introducer into the body lumen to a deployment location;
deploying said endoluminal device from said introducer and into the body lumen, wherein said endoluminal device expands to form a radial expanded portion; and
imparting a radially outward force against said barb assembly to cause said barb assembly to arc radially outwardly and cause said hook to engage said body lumen.
